(19) United States
(12) Patent Application Publication
(10) Pub. No.: US 2022/0023512 A1
(43) Pub. Date: Jan. 27, 2022

(54) Exhalation Disposal System

(71) Applicant: Carl Morgan, Bowling Green, KY (US)

(72) Inventor: Carl Morgan, Bowling Green, KY (US)

(21) Appl. No.: 17/070,187

(22) Filed: Oct. 14, 2020

Related U.S. Application Data
(60) Provisional application No. 63/054,302, filed on Jul. 21, 2020.

(51) Int. Cl. A61M 1/00; A61M 16/20; A61M 16/04; A61M 39/10

(52) U.S. Cl. CPC A61M 1/0052; A61M 16/208; A61M 2205/0205; A61M 39/105; A61M 16/049

(57) ABSTRACT

The present invention relates to an exhalation disposal system for collecting, transporting and properly disposing of a bio-waste exhalation from an infectious patient in a closed system, thereby minimizing, if not completely eliminating, contact between medical personnel and the infectious patient's bio-waste exhalation. The system utilizes a length of flexible vacuum tubing in communication with both a specialized patient mouthpiece and a waste drain, and a check valve positioned along the length of the tubing to prevent backflow of the bio-waste exhalation. In an exemplary embodiment, the disposal system further comprises at least one sensor, a window and an access point for clearing blockages that may arise in the disposal system during use. The disposal system is configured to service multiple infectious patients simultaneously.

### CROSS-REFERENCE TO RELATED APPLICATION

The present application claims priority to, and the benefit of, U.S. Provisional Application No. 63/054,302, which was filed on Jul. 21, 2020, and is incorporated herein by reference in its entirety.

### BACKGROUND

The present invention relates generally to the field of medical biological waste management systems. More specifically, the present invention relates to a bio-waste exhalation disposal system having an improved medical mouthpiece used for transporting bio-waste exhaled from an infected patient directly to a lavatory drain for proper and sanitary disposal. The exhalation disposal system includes a length of flexible vacuum tubing that passes through a medical mouthpiece to reach the drain. The exhalation disposal system provides minimal exposure to medical professionals treating patients infected with dangerous illnesses and diseases, and provides for immediate containment, transit and disposal of diseased air, fluid and other waste from patients. The exhalation disposal system allows the diseased or contaminated exhalation to be eliminated from the patient and transported directly to the drain for proper disposal, thereby protecting healthcare workers from coming into contact with the same. Accordingly, the present specification makes specific reference thereto. Nonetheless, it is to be appreciated that aspects of the present invention are also equally amenable to other like applications, devices and methods of manufacture.

By way of background, medical personnel treating patients infected with dangerous illnesses and infectious diseases are at constant risk of exposure to the same. Communicable diseases, such as COVID-19, are predominantly contracted through contact with infected individuals or droplet transmission attributed to relatively large respiratory particles, which are subject to gravitational forces and travel approximately one meter from the patient. For example, airborne transmission may occur if patient respiratory activity or a medical procedure generates respiratory aerosols, thus heightening the risk of infection to those medical personnel working in close proximity to the infected patient. In addition to respiratory aerosols, bio-waste exhaled by the patient can also cause contamination in the work areas of medical professionals, visitors, staff, and the like. Bio-waste exhaled by a patient may include, but is not limited to, body fluids, blood, pathological waste, gaseous and vapor matters, and the like.

Currently, medical staff and other personnel wear Personal Protective Equipment (PPE) to protect themselves from infectious diseases, and to reduce risk of viral transmission via contact, droplets or airborne particles. By way of background, PPE refers to protective clothing, helmets, gloves, face shields, goggles, face masks, respirators and the like that are designed to protect the wearer from injury or the spread of infection or illness. More specifically, PPE acts as a barrier against infectious materials, such as viral and bacterial contaminants, and typically protects a healthcare worker's skin, mouth, nose, eyes, etc. Unfortunately, PPE is not always 100% effective against such bacteria, germs, viruses, airborne pathogens and the like, and medical professionals and other workers may still contract infectious diseases from patients with dangerous diseases, even while wearing PPE.

Additionally, bio-waste exhalation from a patient is oftentimes difficult to safely capture and transport from one place to another, such as from the infected patient to a proper bio-waste disposal site. More specifically, there is a relatively high risk of contact between the medical personnel and the infected patient and/or bio-waste exhalation during the bio-waste collection, transport and disposal processes, as manual effort, potential physical contact and use of containers are required for the proper movement, transport and disposal of patient exhalation waste. These types of infectious waste may also contain pathogenic micro-organisms, which are easily transmitted to medical personnel by absorption through mucous membranes, inhalation, ingestion and/or other related pathways.

Additionally, if not properly collected and disposed of, bio-waste exhalation can enter the air stream and contaminate surfaces and affect those breathing in the ambient air. For example, research has shown that air samples collected from intensive care unit rooms, general wards, clinical observation rooms, visitation areas and the like have exhaled breath condensate samples of disease, such as COVID-19, from the infected patients. Based on studies of the various coronaviruses, it is estimated that infected patients exhale 0 to 100,000 virus particles per 30 minutes, or as many as 4,800,000 particles per day.

In an effort to reduce such risk, some hospitals and other medical facilities will utilize a scavenger system to gather or collect gas or aerosolized medication after it is exhaled from the patient. However, such systems are individualized, expensive and may not adequately protect medical and other personnel from coming into contact with contaminated air and/or gases exhaled from a patient. In fact, no commercially available system currently removes the exhalation waste from an infectious patient through a closed drainage system. Some commercially available drainage systems were originally developed to avoid open drainage by enclosing the drainage through the catheter into a receptacle. The receptacle in all such systems is periodically emptied in an open environment, thereby exposing the entire system and the surrounding area to potential contamination.

Therefore, there exists a long felt need in the art for a bio-waste exhalation disposal system that provides minimal exposure to a medical provider while disposing of exhaled waste from a patient. There is also a long felt need in the art for a bio-waste exhalation disposal system that transports bio-waste exhalation from an infected patient directly to a lavatory drain without manual intervention by a medical staff. Additionally, there is a long felt need in the art for a bio-waste exhalation disposal system that utilizes a transporting system connected to a mouth cover, such as a mouthpiece for a patient, which leads the bio-waste directly to a sewer inlet. Moreover, there is a long felt need in the art for a bio-waste exhalation disposal system which is cost-efficient, easily installed, and that can be integrated with the existing infrastructure of a hospital or other healthcare facility. Finally, there is a long-felt need in the art for a bio-waste exhalation disposal system that improves safety for medical personnel treating patients with infectious diseases, is relatively inexpensive to manufacture, and is safe and easy to use.

The subject matter disclosed and claimed herein, in one embodiment includes a bio-waste exhalation disposal system that is designed to enhance patient exhalation, while containing, transporting, and properly disposing of exhaled waste. The bio-waste exhalation disposal system is preferably comprised of a mouthpiece that is in fluid communication with a flexible vacuum that is, in turn, in fluid communication with a custom PVC fitting at a sewer or drain line inlet. The mouthpiece and flexible vacuum tube may also include a check valve, and an anti-microbial coating or, alternatively, may be manufactured with the anti-microbial material. When not in use, the mouthpiece can be removed from the patient, and a cork or other sealing agent (e.g. a rubber stopper, film or the like) can be installed in the vacuum line.

In this manner, the bio-waste exhalation disposal system of the present invention accomplishes all of the forgoing objectives and provides a relatively safe, easy, convenient and cost-effective solution for safely collecting, transporting and properly disposing of the exhaled waste of an infectious patient, while minimizing the risk of exposure to medical and other personnel.

### SUMMARY

The following presents a simplified summary in order to provide a basic understanding of some aspects of the disclosed innovation. This summary is not an extensive overview, and it is not intended to identify key or critical elements or to delineate the scope thereof. Its sole purpose is to present some general concepts in a simplified form as a prelude to the more detailed description that is presented later.

The subject matter disclosed and claimed herein, in one embodiment thereof, comprises a unique exhalation disposal system for gathering, transporting and disposing exhaled waste from an infectious patient. The exhalation disposal system comprises a medical mouthpiece to be worn by a patient, a flexible vacuum pipe connected to the medical mouthpiece on one end and a lavatory drain on a second end via a custom PVC fitting at a drain inlet.

In a further embodiment of the present invention, a unique bio-waste exhalation disposal system is disclosed and comprises a patient mouthpiece worn by a patient, a flexible vacuum hose connected to the patient mouthpiece one a first end and having a second end connected to a PVC fitting that is, in turn, attached to a sewer inlet. The pressure difference between the lower pressure sewer inlet, and the higher-pressure hospital air draws exhalation waste from the patient's mouthpiece into the sewer for proper disposal. More specifically, the vacuum hose provides a path from the patient's mouthpiece to the sewer to dispose of the exhaled waste without restriction.

In a further embodiment of the present invention, an exhalation disposal system designed to enhance patient exhalation and the capturing, transporting and disposing of the exhaled waste, all while also using the central air conditioning system deployed in a hospital or other treatment facility is disclosed. More specifically, the exhalation disposal system uses a straight-line channel along the vacuum lines of a central air conditioning system descending from a patient's bed level to a sewer entrance, which provides positive containment, movement, and disposal of the patient's exhalation waste.

In a further embodiment of the present invention, the exhalation disposal system is designed with one sewer inlet having an inner diameter of approximately 1½″ to simultaneously draw exhaled waste from up to six infectious patients in fluid with a flexible vacuum hose and up to six ⅝″ mouthpieces. After being fitted with a custom mouthpiece attached to the vacuum line leading to the sewer inlet of the present invention, the up to six infectious patients will experience immediate containment, transit and disposal of diseased air, fluid and other waste emanating therefrom. The exhalation disposal system of the present invention provides for minimal exposure, if any, of the medical provider to the exhaled waste.

In a further embodiment of the present invention, a method for non-invasive patient exhalation disposal is disclosed. The method comprises an infectious disease patient wearing a custom mouthpiece that is fitted with a flexible vacuum line with an inner diameter of ⅝″ or more, connecting the vacuum line to a custom PVC fitting attached to a sewer or drain inlet and transporting the exhaled contents from the patient to the sewer or drain through a straight path without any interaction with medical professionals. Once the exhaled contents are disposed of safely, the mouthpiece is removed, and a cork, stopper or other seal is positioned over the inlet opening and installed therein.

In a still further embodiment of the presently described invention, a containment, transit and disposal system for exhaled waste from an infectious patient is described. The disposal system comprises a custom mouthpiece fitted with a flexible vacuum line having an interior diameter of ½″ or more connected to a vacuum line, which is in turn connected to a custom PVC fitting attached to a sewer or drain inlet, wherein the sewer or drain inlet is separate from the entrance for bedside urinals and other extraction devices.

In a yet further embodiment of the present invention, a treatment system for respiratory illness is disclosed and comprises a negative pressure system for circulating air flow and withdrawing aspirants and other exhalents from an infectious patient. More specifically, a disposable flexible hose and mouthpiece is provided for collecting exhalents and aspirants from an infectious patient. The hose has at least one sensor, and is connected to an existing drain line in a treatment room, wherein the drain line has a one-way check valve to prevent the return of material from a drain system.

The containment, transit and disposal system for exhaled waste of the present invention is particularly advantageous as installation costs are minimal, and operating costs are negligible for the use of such systems. Additionally, the system minimizes contact between medical personnel, maintenance staff, visitors, other personnel and infectious patients during bio-waste removal, and improves the safety in hospital areas for those treating people with infectious diseases and illnesses. The system works well with exhaled vapors, fluids, and other waste without restriction, and may also be installed using existing central air conditioning systems, sewer systems, extraction systems, drainage pipes, and the like.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective and partially exploded view of one potential embodiment of the bio-waste exhalation disposal system of the present invention in accordance with the disclosed architecture;

FIG. 2 illustrates a diagrammatic view of one possible arrangement of the various components of the bio-waste exhalation disposal system of the present invention in accordance with the disclosed architecture.

FIG. 3 illustrates a diagrammatic representation of one possible arrangement of a bio-waste exhalation disposal circuit of the present invention in accordance with the disclosed architecture; and

FIG. 4 illustrates a diagrammatic representation of one possible method of using the bio-waste exhalation disposal system of the present invention to draw exhaled air and other aspirants from an infectious patient in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a patient bio-waste exhalation disposal system that provides minimal exposure to a medical provider while disposing of exhaled waste from an infectious patient, and that transports bio-waste exhalation from the patient directly to an appropriate disposal location (e.g., a lavatory drain) without manual intervention by medical or other personnel. Additionally, there is a long felt need in the art for a bio-waste exhalation disposal system that utilizes a transporting system connected to a mouth cover, such as a mouthpiece for a patient, which leads the bio-waste directly to the inlet of the drain in an entirely closed system. Moreover, there is a long felt need in the art for a bio-waste exhalation disposal system that is cost-efficient, easy to install, and that can be integrated into the existing infrastructure of the hospital or other medical facility. Finally, there is a long-felt need in the art for a bio-waste exhalation disposal system that improves safety for the medical personnel treating patients with infectious diseases, is relatively inexpensive to manufacture, and is safe and easy to use.

The present invention, in one exemplary embodiment, is an exhaled bio-waste containment, transit and disposal system comprised of an improved medical mouthpiece used for transporting bio-waste exhalation from an infectious patient directly to a lavatory drain in an entirely closed system. More specifically, the disposal system comprises a length of flexible vacuum tubing that is in fluid, one way communication with both the medical mouthpiece and an inlet on the lavatory drain, wherein the exhalants and the aspirants from the infectious patient are only permitted to flow in the direction or the drain and not vice versa. The disposal system provides minimal exposure to medical professionals treating patients infected with dangerous illnesses and diseases, and provides for immediate containment, transit and disposal of diseased air, fluid and solid waste from the patient.

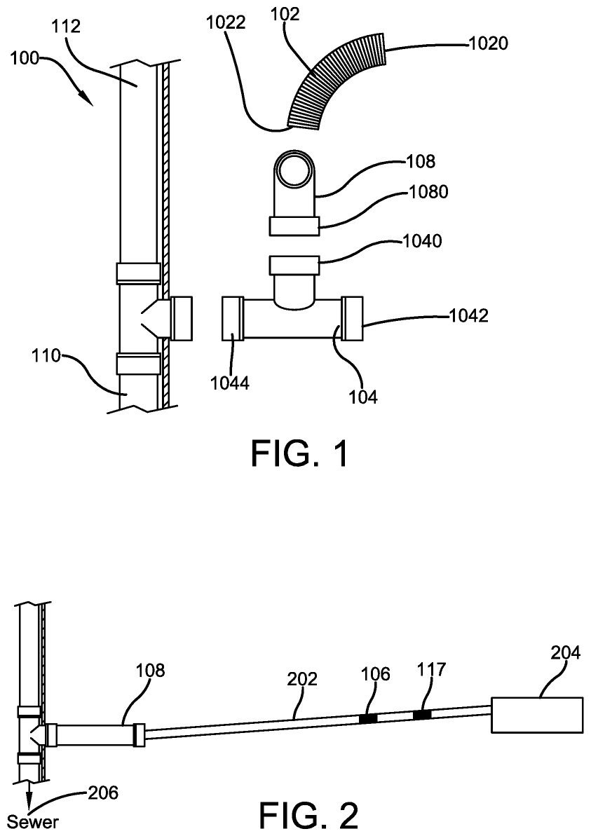

Referring initially to the drawings, FIG. 1 illustrates a perspective and partially exploded view of one potential embodiment of the bio-waste exhalation disposal system 100 of the present invention in accordance with the disclosed architecture. More specifically, the bio-waste exhalation disposal system 100 is comprised of a customizable mouthpiece 300 to be worn by a patient suffering from an infectious disease and that is in fluid communication with a first end 1020 of a flexible vacuum hose 102. An opposite or second end 1022 of the hose 102 is in fluid communication with a compatible and custom PVC fitting 108, which in turn is in fluid communication with a PVC tee fitting 104. The PVC tee fitting is comprised of three openings, namely a right end 1042, an opposing and in line left end 1044, and an opening 1040 positioned approximately midway between, and perpendicular to, the right and left ends 1042, 1044.

In one embodiment, the hose 102 can be a PVC pipe or a rubber pipe. However, the flexible tubing or hose 102 may also be transparent, or have a viewing window 106 therein so that the medical professionals and other treatment personnel can see the waste material passing through the hose 102. The flexible tubing or hose 102 may also comprise an access point to allow the hose 102 to be unclogged with dismantling the entire system 100.

The tee fitting 104 allows a single line to be split into two separate lines with a 90-degree connection, or the tee fitting 104 can be used to connect two lines into one main line. Also, a second end 1080 of the custom fitting PVC 108 has a slot to be fitted into slot 1040 of the PVC 104 at an approximately 90-degree angle. The end 1044 of the PVC 104 may be fixed to a pipe that extends through a wall or other static structure and is connected to a sewer entrance so that the waste exhaled by the patient wearing the mouthpiece travels through the hose 102 in a unilateral direction and into the sewer or drain 110. More specifically, the end 1042 of the PVC 104 is connected to a drain 110 to draw the exhalation waste thereto, and the hose 102 enables a path from the mouthpiece 301 to the drain 110 to conduct exhaled waste without restriction.

The sewer line 110 may also include a vent 112. More specifically, the vacuuming force provided by the lower pressure of the sewer 110 and vent 112 combination and the relatively higher-pressure air flow of, for example, the hospital's HVAC system will draw exhalation air from the patient and drive the exhaled air to the sewer or drain 110. In this manner, the system 100 removes diseased vapors, fluids, and other waste from the patient in a unilateral direction (i.e., no backflow), without restriction, and without a need for contact between medical personnel, support staff and the like and the infectious patients during bio-waste removal process. In one embodiment, the flexible vacuum hose 102 may have an interior diameter of 16 mm or more, and a length of at least 4 feet. The system 100 may use or tie into an existing sewer or drain infrastructure, or a new sewer or drain entrance, as per the existing construction and drainage system of the medical facility.

FIG. 2 illustrates a diagrammatic view of one possible arrangement of the various components of the bio-waste exhalation disposal system 100 of the present invention in accordance with the disclosed architecture. More specifically, FIG. 2 illustrates a generally straight-line hose or tube 102 descending from an elevation associated with a patient lying on a bed 204 to a sewer 110. The hose or tube 102 is present and provides positive containment, movement, and disposal of patient exhalation waste. The tube 102 is in fluid communication with both the patient's mouthpiece 301 on one end (i.e., the elevated portion) and the drain 110 on the opposite end (i.e., at a lower elevation).

Additionally, the vacuum lines or pipes 102 with an extrusion coating provide a relatively straight path for the exhaled waste to be transported and disposed of without restriction or contact by others. The arrangement offers a convenient method for addressing the exhalation and aspiration needs of a patient lying on the patient's bed 204 in a non-invasive manner, and without unnecessarily exposing healthcare personnel to the patient's bio-waste. The vacuum tubing 204 is relatively flexible, and may be supported via one or more J channel supports (not shown). In one embodiment, 200-degree semicircle J channel supports are used for supporting vacuum tubing 102 to transport the waste from the patient to the sewer or drain entrance 110. In another embodiment, 160-degree J channel supports are used for supporting vacuum tubing 102 to transport the waste from the patient to the sewer or drain entrance.

Ideally, the vacuum tube 102 descends from the patient level at an angle suitable for easy gravitational movement of the waste within the tube 102. For example, the vacuum tube 102 may have inclination in the range of 30-75 degrees from the floor of the room in which the bed 204 of the patient is placed. Also, as previously stated, the sewer or drain 110 may have a custom PVC fitting 108, which would have a slot therein for securely attaching to the descending tube 102. More specifically, the tubing 102 is fitted into the slot in the PVC fitting 108 and carries the vapor, fluid, and other waste from the mouthpiece 301 of the patient to the drain 110. In one embodiment, the vacuum line 102 may be a flexible vacuum hose that utilizes the lower pressure sewer-vent and the higher-pressure hospital air to draw exhalation from the patient and drive the same toward the sewer drain 110.

As previously stated, the tubing 102 may also include a window 106, which can be used to both view material passing through the flexible tubing 102, and/or as an access point in order to clear a blockage and service the system 100 if necessary. In one embodiment, the disposal system 100 may also include one or more sensors 117 disposed in the flexible tubing 102 and that can detect the presence of bacteria or a virus, as well as an environmental condition such as temperature and humidity. The sensors 117 can also be in communication with a computer or other system (not shown), such as those used to monitor the well-being of the patient. The sensors 117 can transmit information wirelessly to the monitoring system, or may be a visual type of sensor that changes color (or emits an audible sound) depending, for example, on the presence of a particular pathogen or condition, such as temperature or humidity.

The size of the vacuum hose 102 may be selected to accommodate the anticipated quantity and flow rates of the anticipated bio-waste that will be transported thereby, as well as the size of the custom PVC fitting 108 at the entrance of the drain 110. In one embodiment, one sewer or drain 110 inlet having an inside diameter of 1½″ can draw exhaled waste from up to six infectious patients with a vacuum hose 102 having an interior diameter of ½″ and a customized mouthpiece. In another embodiment, one sewer inlet of 2½″ I.D. can draw exhaled waste from up to ten infectious patients with a vacuum hose having an inside diameter of ⅝″ and a customized mouthpiece. Nonetheless, the size of the sewer or drain inlet and vacuum line/hose 102 can be set based on the requirements of the user.

The vacuum hose 102 is relatively lightweight, yet durable, and both the hose 102 and the mouthpiece 301 may be comprised of a disposable, bio-degradable, and/or anti-microbial material. In one embodiment, the flexible hose 102 may be comprised of a sturdy and flexible polyethylene material and can be of a translucent or opaque nature to make the content invisible while transporting to the sewer 110. The material selected for the hose 102 should also have excellent resistance to moisture, chemicals, and abrasives.

The disposal system 100 of the present invention is a splash-free, drip-free, and completely contained system, which alleviates the problem of cross-contamination with other patients and/or medical personnel, thus alleviating the problems caused by exhaled waste from infectious patients. More specifically, the disposal system 100 provides a sealed-flow passageway extending between the mouthpiece 301 worn by the patient, and the entrance to the sewer or drain 110, and may further comprise a check valve 302, as explained more fully below.

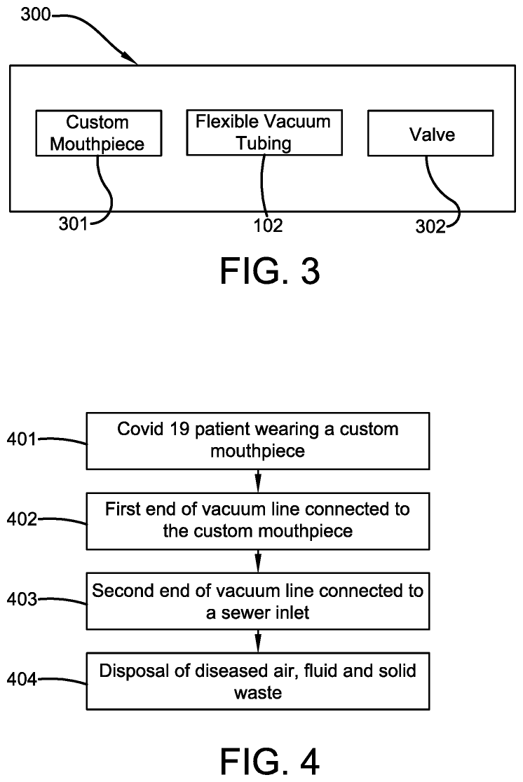

FIG. 3 illustrates a diagrammatic representation of one possible arrangement of a bio-waste exhalation disposal circuit 300 of the present invention in accordance with the disclosed architecture. More specifically, the disposable circuit 300 provides immediate containment, transit and disposal of diseased air, fluids and other wastes exhaled by the patient. The disposable circuit 300 may be marketed and made commercially available as a complete kit comprising a length of flexible vacuum tubing or hose 102, a non-return or check valve 302, and a customizable mouthpiece 301. The valve 302 may include a trap in order to prevent material from entering the circuit 300 or backflowing through the tubing 102 (e.g., in the direction of the patient). The disposable mouthpiece 301 may be molded to fit the opening of the mouth of a specific patient, wherein the patient may seal the mouthpiece 301 by wrapping his or her lips around the same during an exhalation process or procedure. Also, the length of vacuum tube 102 is relatively flexible, and may be of a standard diameter, such that it fits into any standard sewer or drain inlet present at a hospital or other medical facility.

FIG. 4 illustrates a diagrammatic representation of one possible method of using the bio-waste exhalation disposal system 100 of the present invention to draw exhaled air and other aspirants from an infectious patient in accordance with the disclosed architecture. The disposal system 100 acts as a completely contained ventilator system wherein the vacuuming action will assist and encourage the patient's exhalation of the bio-waste in an efficient and safe manner. The method begins at step 401, wherein a patient suffering from an infectious disease, such as COVID-19, installs a mouthpiece 301, wherein the mouthpiece is customizable to fit the specific patient's mouth to provide for an adequate seal. At step 402, a first end of a vacuum line 102 of the present invention is connected to the custom mouthpiece 301. At step 403, the second end of the vacuum line 102 leads, and is attached to, an inlet on a drain or sewer 110 to properly dispose of the patient's bio-waste. At step 404, the system 100 provides immediate containment, transit and disposal of diseased air, fluid and other waste exhaled by the patient. The system enables the medical personnel to provide treatment to, for example, a COVID-19 patient without the risk of becoming infected by the exhaled contents from the infectious patient. As described above, the system 100 may further comprise anti-microbial materials to kill bacteria, germs, viruses and other pathogens, a check valve 302 to prevent backflow to the patient from the drain 110, a sensor 117 to monitor the condition of the system 100, a window 106 for viewing the interior space of the tubing 102 to ensure proper operation, and an access point for clearing any clogs in the tubing 102.

In one embodiment, the system 100 may be implemented using a separate sewer or drain entrance for bedside urinals such as those available from recreational camper suppliers. The separate sewer entrance would be connected through a vacuum hose 102 to draw exhalations from the patient and direct the same to the sewer or drain 110 through the separate sewer entrance.

Thus, according to broad aspect, the present invention provides a system for collecting, transporting and discharging bio-waste to a disposal passageway in a manner that protects medical and other personnel, as well as protecting areas where such persons work and related support devices from contamination. It should be noted that the contaminated waste material from the patient remains securely transported through the flexible vacuum line 102 to the sewer 110 in a closed system.

Thus, the disposal system 100 of the present invention minimizes contact between medical personnel and infected patients during bio-waste exhalation removal. The disposal system 100 also improves safety in hospitals and other medical centers, and is cost-effective and user-friendly with minimal installation costs and negligible operating costs. The disposal system 100 provides a closed drainage system in which gaseous exhalations from the patient are purged without exposure to the ambient atmosphere.

Certain terms are used throughout the following description and claims to refer to particular features or components. As one skilled in the art will appreciate, different persons may refer to the same feature or component by different names. This document does not intend to distinguish between components or features that differ in name, but not structure or function. As used herein “bio-waste exhalation disposal system”, “exhalation disposal system”, “patient exhalation disposal system”, and “containment, transit and disposal system” are interchangeable and refer to the bio-waste exhalation disposal system of the present invention.

Notwithstanding the forgoing, the bio-waste exhalation disposal system 100 of the present invention can have components of any suitable size and configuration as are known in the art without affecting the overall concept of the invention, provided that it accomplishes the above stated objectives. One of ordinary skill in the art will appreciate that the size, configuration, arrangement and material of the components of the bio-waste exhalation disposal system 100 as shown in the FIGS. are for illustrative purposes only, and that many other arrangements, sizes and shapes of the bio-waste exhalation disposal system 100 are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

1. A kit for retrofitting a ventilation system comprising:
a mouthpiece sized and configured for an individual patient, the mouthpiece having a connection fitting;
an extraction hose having a first end and a second end, wherein the first end is connected to the mouthpiece;
a valve for controlling a flow of air and a flow of aspirants within the extraction hose; and
a drain line having a first end, a second end, and a fitting positioned on the first end of the drain line, wherein the extraction hose extends from the mouthpiece to the fitting on the drain line.

2. The kit for retrofitting a ventilation system of claim 1, wherein the mouthpiece is disposable.

3. The kit for retrofitting a ventilation system of claim 1, wherein the extraction hose is a flexible hose.

4. The kit for retrofitting a ventilation system of claim 1 further comprising at least one tee joint for connecting one or more patients to the drain line.

5. The kit for retrofitting a ventilation system of claim 1, wherein the valve is a one-way valve.

6. The kit for retrofitting a ventilation system of claim 5, wherein the one-way valve comprises a trap.

7. The kit for retrofitting a ventilation system of claim 1, wherein each of the valve, the drain line, the fitting and the connection fitting are constructed of a PVC material.

8. The kit for retrofitting a ventilation system of claim 1, wherein each of the mouthpiece and the extraction hose are comprise of an antimicrobial material.

9. The kit for retrofitting a ventilation system as recited in claim 1, wherein the extraction hose comprises an access point for clearing the extraction hose of a blockage.

10. A ventilation system comprising:
a patient treatment facility comprised of an HVAC system;
a treatment room in communication with the HVAC system, wherein the treatment room comprises a patient ventilation system and a drain; and
a discharge system for discharging an aspirant from a patient, wherein the discharge system is in fluid communication with the drain and the discharge system utilizes the HVAC system to create a negative pressure to withdraw the aspirant from the patient and discharge the aspirant to the drain.

11. The ventilation system of claim 10, wherein the discharge system is comprised of a disposable mouthpiece, and a length of flexible hose in fluid communication with each of the disposable mouthpiece and the drain.

12. The ventilation system of claim 10, wherein the discharge system comprises a one-way valve that prevents the flow of a substance from the drain to the patient.

13. The ventilation system of claim 10, wherein the discharge system is constructed from a PVC material.

14. The ventilation system of claim 10, wherein the discharge system further comprises an anti-microbial material.

15. The ventilation system of claim 11, wherein the length of flexible hose comprises a window and an access point for accessing a blockage in the length of flexible hose.

16. The ventilation system of claim 10, wherein the discharge system further comprises at least one sensor for detecting at least one of a bacteria, a virus, a temperature and a humidity.

17. The ventilation system of claim 16, wherein the at least one sensor is connected to a system for monitoring the patient.

18. A treatment system for respiratory illness comprising:
a negative pressure system for circulating an air flow and for withdrawing an aspirant from a patient;
a hose and a mouthpiece for collecting the aspirant from the patient, wherein the hose comprises at least one sensor and is in fluid communication with the mouthpiece; and
a drain line in a treatment room in fluid communication with the hose, wherein the drain line is comprised of a one-way valve to prevent a return of the aspirant to the patient after it enters the drain line and passes through the one-way valve.

19. The treatment system for respiratory illness of claim 18, wherein the hose further comprises a window and an access point for clearing a blockage in the hose.

20. The treatment system for respiratory illness of claim 18, wherein the at least one sensor detected at least one of a bacteria, a virus, a temperature and a humidity.
